(19) United States
(12) Patent Application Publication
(10) Pub. No.: US 2007/0293923 A1
(43) Pub. Date: Dec. 20, 2007

(54) LEAD WITH ORIENTATION FEATURE

(75) Inventors: Brian D. Soltis, St. Paul, MN (US); Mitchell A. Smith, Mahtomedi, MN (US); Bruce A. Tockman, Scandia, MN (US)

Correspondence Address: FAEGRE & BENSON, LLP;BOSTON SCIENTIFIC PATENT DOCK, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/424,440

(22) Filed: Jun. 15, 2006

(51) Int. Cl. A61N 1/05

(52) U.S. Cl. 607/122; 607/125

(57) ABSTRACT

A left ventricular lead is provided for placement in a branch vessel of the coronary sinus, the vessel having a vessel wall and an adjacent myocardium. The lead includes a lead body having a central lumen extending therethrough, at least a first electrode on the lead body and at least a first orientation feature protruding from the lead body for orienting one or more of the electrodes into contact with the myocardium. The lead may also include a pre-shaped curvature. The orientation feature may also aid in steering the lead into a selected branch vessel of the coronary sinus and in fixing the lead within the branch vessel.

### TECHNICAL FIELD

The present invention relates to medical devices and methods for accessing an anatomical space of the body. More specifically, the invention relates to devices and methods for orienting a lead within a branch of the coronary sinus.

### BACKGROUND

Implantable medical devices for treating irregular contractions of the heart with electrical stimuli are well known in the art. Some of the most common forms of such implantable devices are defibrillators and pacemakers. Various types of electrical leads for defibrillators and pacemakers have been suggested in the prior art.

A broad group of leads may be characterized by the fact that they are placed transvenously. These leads are introduced into the patient's vasculature at a venous access site and travel through veins to the locations where the leads' electrodes will implant in or otherwise contact coronary tissue. One large subfamily of the group of transvenously-placed leads are those that are implanted in the endocardium (the tissue lining the inside of the heart) of the right atrium or ventricle. Another subfamily of the group of transvenously-placed leads are those that are placed in the branch vessels of the coronary venous system to stimulate the left ventricle.

The treatment of heart failure often requires left ventricular stimulation either alone or in conjunction with right ventricular stimulation. For example, cardiac resynchronization therapy (also commonly referred to as biventricular pacing) is an emerging treatment for heart failure, which requires stimulation of both the right and the left ventricle to increase cardiac output. Left ventricular stimulation requires placement of a lead in or on the left ventricle in the lateral or posterior-lateral aspect/region of the heart. One technique for left ventricular lead placement is to advance a lead endovenously into the coronary sinus and then advance the lead through a branch vein onto the surface of the left ventricle. Although methods and tools have been developed to navigate the lead through the vasculature, and in particular to direct the lead into a selected branch vessel of the coronary sinus, it can be difficult to orient the electrodes to face and make contact with the myocardium.

The left ventricle beats forcefully as it pumps oxygenated blood throughout the body. Repetitive beating of the heart, in combination with patient movement, can sometimes dislodge the lead from the myocardium. Over time, the electrodes may lose contact with the heart muscle, or move from their original location and orientation. If the electrodes come into contact with the branch vessel wall, rather than the myocardium of the left ventricle, a degraded site for sensing and pacing will result.

What is needed, then, is an improved lead and method of implantation for orienting the lead into the coronary sinus branch vessels and for orienting the lead electrodes into contact with the myocardium.

### SUMMARY

In one embodiment, the present invention is a left ventricular lead for placement in a branch vessel of the coronary sinus. In general, the branch vessel has a vessel wall and is adjacent the myocardium. The lead includes a lead body having a lumen, a first electrode on the lead body, and an orientation feature protruding from the lead body for orienting the electrode into contact with the myocardium.

In another embodiment, the present invention is a left ventricular lead for placement in a branch vessel of the coronary sinus. In general, the branch vessel has a vessel wall and is adjacent the myocardium. The lead includes a lead body having a lumen, a first electrode on the lead body, and an orientation feature protruding from the lead body for orienting a distal tip of the lead body into a selected branch vessel.

In yet another embodiment, the present invention is a method of implanting a lead in a selected branch vessel of the coronary sinus that is adjacent a myocardium. A lead body is provided that has a lumen extending therethrough, at least a first electrode on the lead body, and at least a first orientation feature protruding from the lead body for orienting one or more the electrodes into contact with the myocardium. The lead body is advanced through the coronary sinus and into the selected branch vessel. The at least first orientation feature is engaged against a wall of the branch vessel opposite the at least first electrode.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic drawing of a cardiac rhythm management system including a pulse generator coupled to a lead deployed in a patient's heart according to one embodiment of the present invention.

FIG. 2 shows a side sectional view of a portion of a lead according to one embodiment of the present invention.

FIG. 3 shows a side schematic view of the lead of FIG. 2 accessing a branch vessel of the coronary sinus.

FIG. 4 shows a side schematic view of a portion of a lead according to another embodiment of the present invention.

FIG. 5 shows a side schematic view of the lead body of FIG. 4 disposed within a branch vessel of the coronary sinus.

FIG. 6 shows a side schematic view of a portion of a lead accessing a branch vessel of the coronary sinus according to another embodiment of the present invention.

FIG. 7 shows a side schematic view of a portion of a lead according to another embodiment of the present invention.

FIG. 8 shows a side schematic view of a portion of a lead according to another embodiment of the present invention.

FIG. 9 shows a side schematic view of the lead of FIG. 8 accessing a branch vessel of the coronary sinus.

FIG. 10 shows a side schematic view of a portion of a lead in a collapsed configuration according to another embodiment of the present invention.

FIG. 11 shows a side schematic view of the lead of FIG. 10 in a deployed configuration.

FIG. 12 shows a side schematic view of a portion of a lead according to another embodiment of the present invention.

FIG. 13 shows a side schematic view of a portion of a lead according to another embodiment of the present invention.

FIG. 14 shows a side schematic view of a portion of a lead according to another embodiment of the present invention.

FIG. 15 shows a side schematic view of the lead of FIG. 14 accessing a branch vessel.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

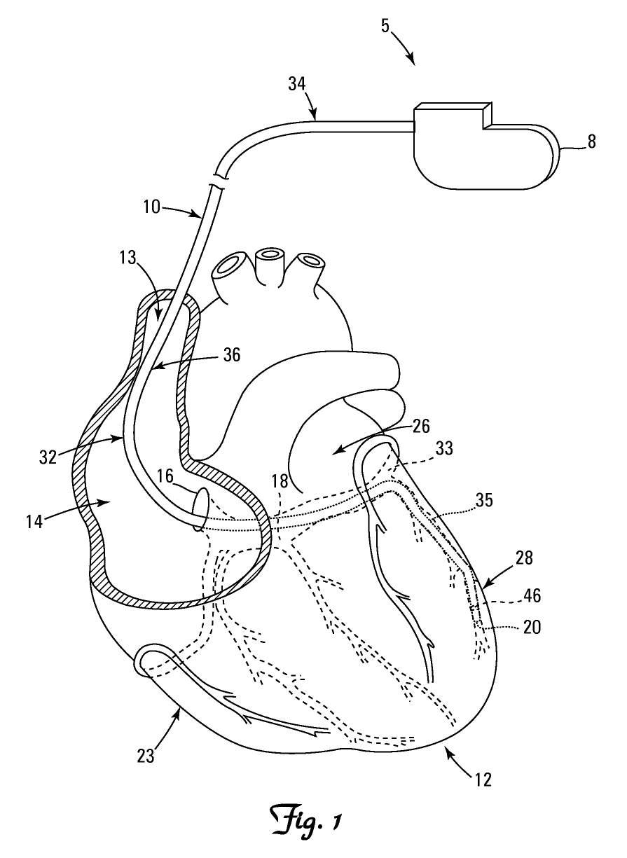

FIG. 1 is a schematic drawing of a cardiac rhythm management system 5 including a pulse generator 8 coupled to a lead 10 deployed in a patient's heart 12 from a superior vena cava 13. As shown, the heart 12 includes a right atrium 14 and a right ventricle 23, a left atrium 26 and a left ventricle 28, a coronary sinus ostium 16 in the right atrium 14, a coronary sinus 18, and various cardiac vessels including a great cardiac vein 33 and other branch vessels off the coronary sinus 18 including an exemplary branch vessel 35.

As shown in FIG. 1, the lead 10 includes an elongate body 32 defining a proximal portion 34 and a distal portion 36. The distal portion 36 includes at least one electrode 46 and terminates in a distal tip 20. The proximal portion 34 is operable to manipulate the distal portion 36 through the vasculature to position the distal tip 20 into a branch vessel of the coronary sinus 18.

In the embodiment illustrated in FIG. 1, the distal portion 36 is guided through the superior vena cava 13, the right atrium 14, the coronary sinus ostium 16, and the coronary sinus 18, and into the branch vessel 35 of the coronary sinus 31, with the distal tip 20, and thus the electrode 46, positioned within the branch vessel 35. The illustrated position of the lead 10 may be used, for example, for sensing physiologic parameters and delivering a pacing and/or defibrillation stimulus to the left side of the heart 12 at a myocardium 24 of the heart 12. Additionally, it will be appreciated that the lead 10 may also be partially deployed in other cardiac vessels such as the great cardiac vein 33 or other branch vessels for providing therapy to the left side (or other portions) of the heart 12.

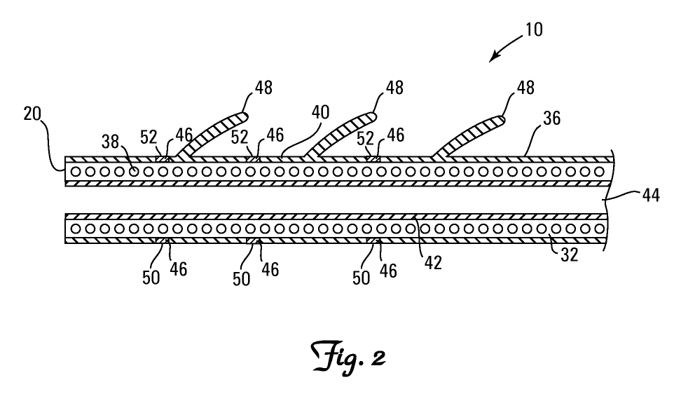

FIG. 2 is a detailed sectional view of the distal portion 36 of the electrical lead 10 according to one embodiment of the present invention. The lead body 32 is constructed of a conductive coil 38 sandwiched between an outer sheath 40 and an inner sheath 42. The inner sheath 42 defines a lumen 44 that is open at the distal tip 20. One or more electrodes 46 are positioned along the lead body 32 and are in electric communication with the coil 38 or other conductors. In the illustrated embodiment, the lead 10 has three electrodes 46. However, in other embodiments, the lead 10 may include fewer or greater electrodes 46. The lead 10 may be bipolar or unipolar. The lead 10 may include multiple spaced apart electrodes having the same polarity. Furthermore, a lead in accordance with the present invention is not limited to the configuration previously described. Rather, the lead 10 may have any configuration as is known in the art. For example, the lead may include a fewer or greater number of lumens 44, a non-coiled conductor 38, multiple conductors 38 in electric communication with separate electrodes 46, or other features as are known in the art.

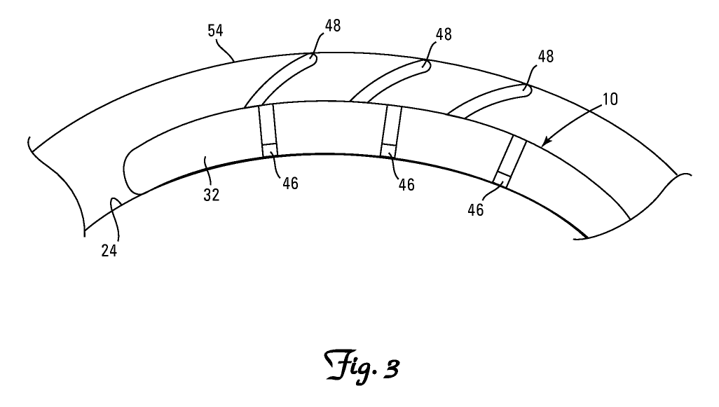

The lead 10 further includes one or more orientation features 48 protruding from the lead body 32. The orientation features 48 are configured and located on the lead body 32 to direct or orient the electrodes 46 into contact with the myocardium 24. As shown in FIG. 3, the orientation features 48 engage a vessel wall 54 opposite the myocardium 24, orienting the electrodes 46 into contact with the myocardium 24. The electrodes 46 may be partially insulated with an exposed electrode surface 50 directly opposite the orientation feature 48 and an insulated surface 52 adjacent the orientation feature 48.

In the illustrated embodiment, there is a one to one relationship between the number of electrodes 46 and the number of orientation features 48. However, the invention is not so limited. Rather, orientation features 48 may be provided in greater or fewer number than electrodes 46. In addition, in the illustrated embodiment, the orientation features 48 are located opposite the lead body 32 from respective electrodes 46. However, the orientation features 48 need not be positioned opposite an electrode 46. Rather, the orientation feature 48 may be positioned at any location along the lead body 32, which, when accessing a branch vessel, engages the vessel wall 54 and tends to orient one or more of the electrodes 46 into contact with the myocardium 24. Due to the complex shape of the branch vessels, as shown in FIG. 1, an orientation feature 48 may be located proximal or distal relative to the electrode 46 and may be radially displaced from the electrode 46 in order to orient the electrode 46 into contact with the myocardium 24. For example, in one embodiment of the invention, the orientation feature 48 is radially offset from the electrode 46 by about 120°.

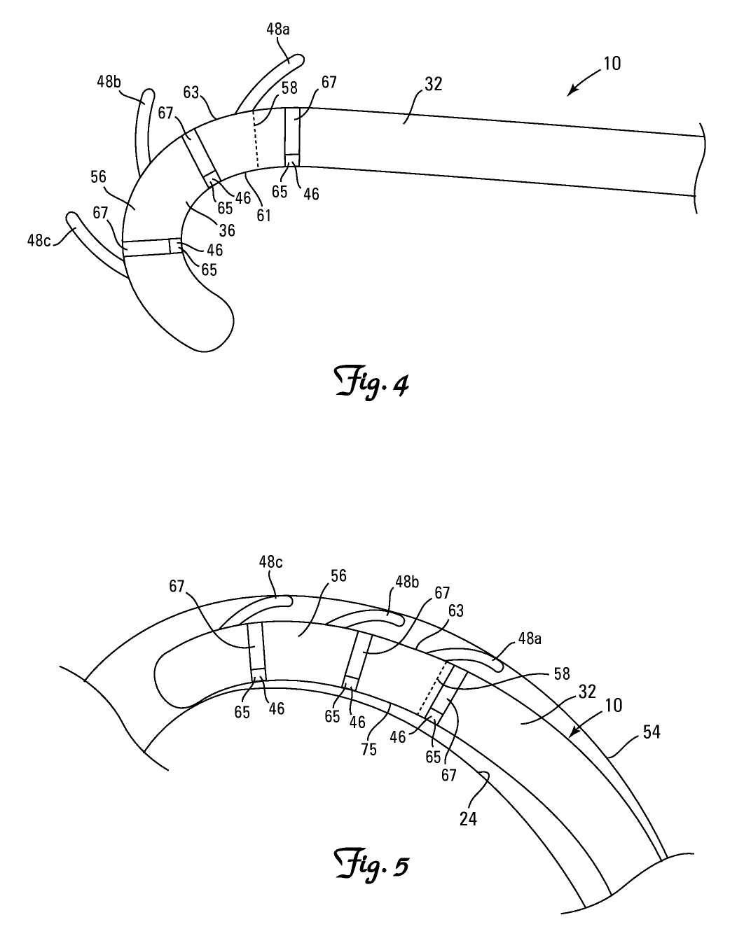

FIGS. 4 and 5 illustrate a portion of the lead 10 according to another embodiment of the invention, in which the distal portion 36 of the lead body 32 is pre-shaped with a curvature 56. The lead body 32 includes one or more inflection regions 58. As used herein, “inflection region” refers to a region of the lead body 32 where the pre-shape of the lead body 32 changes. In the illustrated embodiment, the pre-shape of the lead body 32 changes from generally straight to curved at inflection region 58. In other embodiments, however, the inflection region 58 may be a location on the lead body 32 where the curvature of the lead body 32 changes from a first curvature to a second curvature or where the curvature changes direction.

The orientation features 48a-c are located on the curvature 56 and positioned on the lead body 32 to orient the electrodes 46 into contact with the myocardium 24 rather than the vessel wall 54. As previously discussed, the orientation feature 48a-c may be displaced along the lead body 32 from the corresponding electrodes 46. For example, in the illustrated embodiment, the orientation feature 48a is located on the lead body 32 adjacent the inflection region 58 of the lead curvature 56 rather than opposite the corresponding electrode 46. Locating the orientation feature 48a thusly, in combination with the geometry of the branch vessel and the curvature 56 of the lead body 32, may provide improved contact between the electrode 46 and the myocardium 24.

In the illustrated embodiment, the curvature 56 of the lead 10 is generally J-shaped. However, the lead 10 may have other shapes, including spiraled, canted, S-shaped, etc. The shape of the lead 10 also causes the lead 10 to align itself with the curvature of the heart 12 in such a way that a first surface 61 of the lead 10 will tend to be oriented towards the myocardium 24 while a second surface 63 of the lead 10 will tend to be oriented away from the myocardium 24, or towards the vessel wall 54. In general, then the first surface 61 of the lead 10 will be oriented to contact the myocardium 24 while the second surface 63 of the lead 10 will be oriented to never or seldom contact the myocardium 24. In the illustrated embodiment, a first side 65 of the electrode 46 corresponding to the first surface 61 of the lead 10 is exposed while a second side 67 of the electrode 46 corresponding to the second surface 63 of the lead 10 is insulated. In other embodiments, the electrode 46 is a partial electrode which is only located on the first surface 61 of the lead 10. Because the second side 63 of the lead 10 will tend to be oriented away from the myocardium 24, the electrode 46 need not be positioned on the second side 63. This reduces unwanted stimulation of the vessel wall 54.

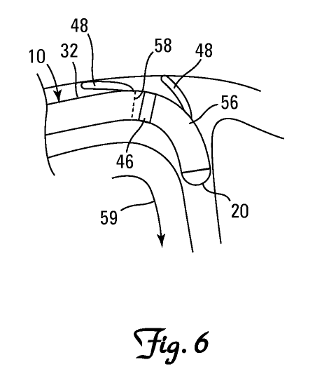

FIG. 6 illustrates a portion of the lead 10 according to another embodiment of the invention, in which one or more of the orientation features 48 are positioned on the lead body 32 to facilitate orienting the distal tip 20 of the lead body 32 into a selected branch vessel, as indicated by arrow 59. The orientation features 48 may be placed at inflection regions 58 of the lead body 32, as shown in FIG. 6, or at other locations on the lead body 32. The lead body 32 may be pre-shaped with a curvature 56, as described with respect to FIGS. 4 and 5, or, in other embodiments, a stylet or guidewire may be inserted into the lead lumen 44 to form the inflection region 58.

In the illustrated embodiment, the orientation feature 48 is located on an outside tangent of the lead curvature 56 at the inflection region 58 of the lead body 32. The orientation feature 48 protrudes from the lead body 32 and orients the distal tip 20 of the lead 10 into the selected branch vessel as the lead 10 is advanced. The location and size of the orientation feature 48 may be adapted to access a vessel having a particular take-off angle. The orientation feature 48 may simultaneously be positioned on the lead body 32 to orient one or more of the electrodes 46 into contact with the myocardium 24 once the lead 10 has accessed the selected branch vessel. In other embodiments, additional orientation features 48 may be provided to serve this purpose. The orientation feature 48 thus serves the dual purpose of aiding in steering the lead 10 through the vasculature and into a selected branch vessel as well as aiding in providing improved contact between the electrodes 46 and the myocardium 24.

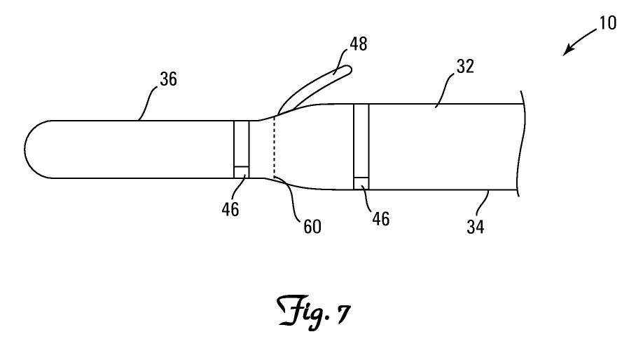

FIG. 7 illustrates a portion of the lead 10 according to another embodiment of the present invention, in which the lead 10 includes one or more flexibility transition regions 60. As used herein, “flexibility transition region” refers to a region of the lead body 32 transitioning from a more flexible portion of the lead body 32 to a less flexible portion of the lead body 32. In general, the lead body 32 is more flexible at the distal portion 36 and less flexible at the proximal portion 34, but this is not always true. In the illustrated embodiment, the flexibility transition region 60 occurs where the lead body 32 reduces in diameter from the proximal portion 34 of the lead body 32 to the distal portion 36. However, in other embodiments, the flexibility transition region 60 may be provided by a change in the material of the lead 10, or in the construction of the lead 10, or by any other means that would cause the flexibility of the lead 10 to vary.

The orientation feature 48 is located on the lead body 32 adjacent to a flexibility transition region 60 of the lead 10. Placing the orientation features 48 at or near the transition region 60 of the lead 10 may aid in orienting the electrode 46 into contact with the myocardium 24 as well as aid in directing, steering or guiding the lead 10 into a desired location.

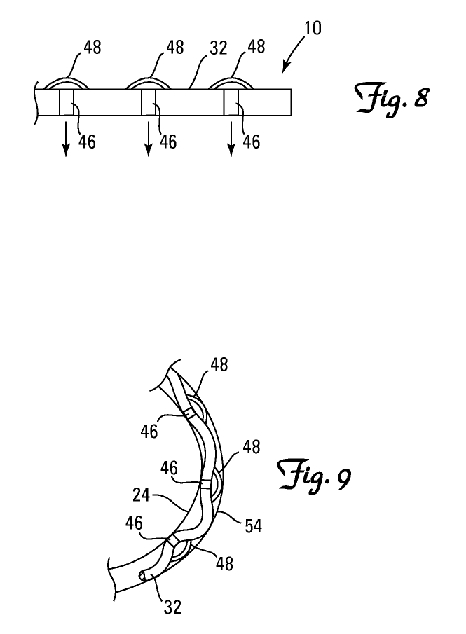

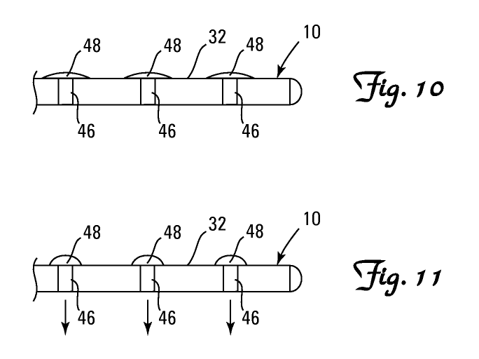

The orientation features 48 may have many configurations and arrangements. FIGS. 8-11 show various embodiments of orientation features 48 according to the present invention. The orientation features 48 may be tines, as shown in FIGS. 2-7, leaf springs, polymer protrusions, expandable members, such as balloons, stents, cages, or other structures formed of nitinol or similar shape memory alloys, etc. The lead 10 may include any number of orientation features 48, and may include combinations of different orientation features 48. For example, FIGS. 8 and 9 show an orientation feature 48 that is a leaf spring, while FIGS. 10 and 11 show an orientation feature 48 that is inflatable or expandable, similar to a stent.

The orientation features 48 may be fixed, as shown with respect to the embodiments generally illustrated in FIG. 2. In other embodiments, the orientation feature may be deployable from a collapsed configuration to a protruding configuration, as shown in FIGS. 8-11. The orientation features 48 may be passively deployable, for example, by being spring loaded or biased, as shown in FIGS. 8 and 9, in which the orientation feature 48 is a leaf spring. In other embodiments, the orientation feature 48 may be actively deployable, for example, by being inflatable, or by being deployed with a tensioning device, a stylet, or other tool. In the embodiment shown in FIGS. 10 and 11, the orientation feature 48 is inflatable or expandable from a collapsed configuration, shown in FIG. 10, to a deployed configuration, as shown in FIG. 11.

Multiple orientation features 48 may be individually and selectively deployed or activated, may be selectively deployed or activated at different stages as the lead 10 is advanced into the heart 12, and may be de-activated or collapsed to allow the physician to remove or reposition the lead 10.

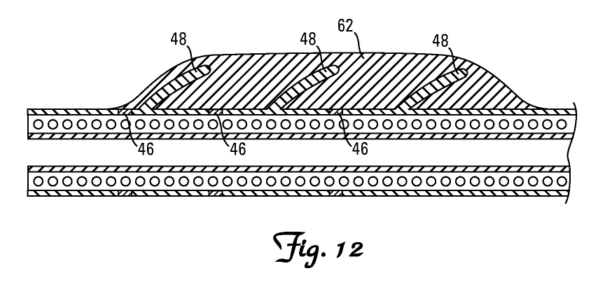

FIG. 12 shows a portion of the lead 10 according to another embodiment of the invention, in which the lead 10 includes a covering or coating 62 over the orientation features 48. The coating 62 may retain the orientation features 48 in a collapsed configuration until the coating 62 is removed. The coating 62 may also be employed to provide the lead 10 with a smooth outer profile whether the orientation feature 48 is fixed, deployable, inflatable or expandable. The coating 62 also allows the lead 10 to be advanced through a catheter. The coating 62 may be dissolvable or water soluble.

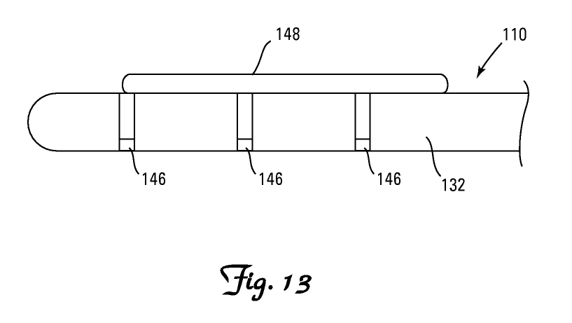

FIG. 13 shows a portion of the lead 110 according to another embodiment, in which the lead 110 includes a single, elongated orientation feature 148. The orientation feature 148 is elongated along the length of the lead body 132 to be opposite or “cover” multiple electrodes 146, providing the orienting functions previously described. In other embodiments, the orientation feature 148 is elongated to cover multiple lead body 132 inflection regions (not shown), transition regions (not shown) or other lead features, or combinations thereof. The orientation feature 148 may be fixed or deployable, and may have any configuration as generally described previously.

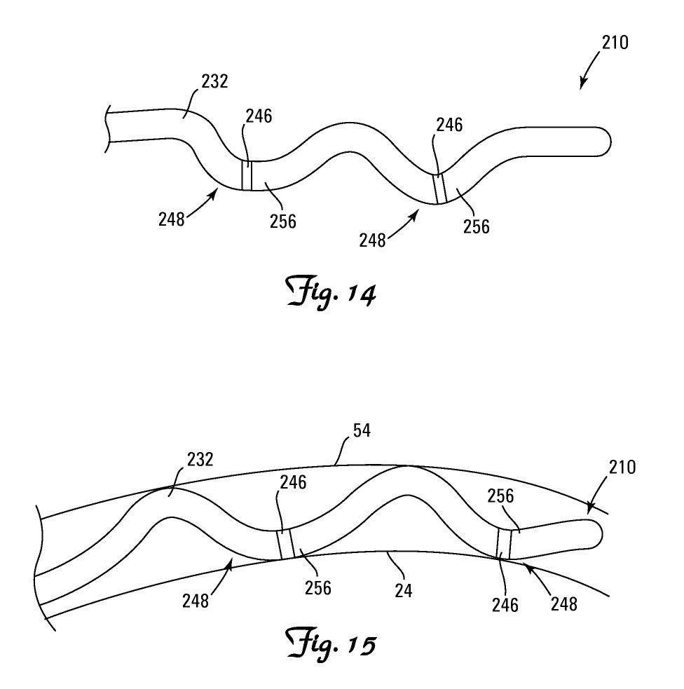

FIGS. 14 and 15 show another embodiment of a lead 210 in which the orientation feature 248 is integrally incorporated into the lead body 232 rather than being a protruding component as shown in the preceding figures. As shown in FIG. 14, the lead body 232 is preshaped with the electrodes 246 residing on an inflection region 256 of the lead body 232 tending to be directed towards the myocardium 24 and away from the branch vessel wall 54. The orientation feature 248 may be incorporated into the lead body 232 through coil shaping, polymer shaping, or both. In other embodiments, the orientation feature 248 may be implemented in combination with the individual orientation features 48 described with respect to FIGS. 2-13.

A lead according to the present invention can provide improved, predictable and preferential contact of the electrodes 46 with the myocardium 24. A lead according to the present invention can provide the ability to direct all of the electrodes on the lead body towards the myocardium. Multiple or redundant electrodes may thus be included on the lead body so that the site for pacing and sensing may be chosen from the preferred location. In addition, the orientation features 48 may be located and configured on the lead body 32 so as to orient the lead body 32 within a desired plane, to fix the lead body 32 at a particular location within a selected branch vessel, or to stabilize the lead body 32 against unwanted rotational movement. The orientation features may also be reversed to allow the physician to re-position or remove the lead. Finally, the orientation feature may limit orientation changes in a chronically implanted lead.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A left ventricular lead for placement in a branch vessel of the coronary sinus, the vessel having a vessel wall and an adjacent myocardium, the lead comprising:
a lead body having a lumen extending therethrough;
at least a first electrode on the lead body; and
at least a first orientation feature protruding from the lead body for orienting one or more of the electrodes into contact with the myocardium.

2. The lead of claim 1 wherein the at least first orientation feature is disposed on the lead body opposite the at least first electrode.

3. The lead of claim 1 wherein the at least first electrode has an exposed surface opposite the at least first orientation feature and an insulated surface adjacent the at least first orientation feature.

4. The lead of claim 1 wherein the at least first orientation feature is one of a tine, a leaf spring, a polymer protrusion, an expandable member, a balloon, a stent, a cage, or a shape memory alloy.

5. The lead of claim 4 further comprising a plurality of orientation features, in which at least one of the orientation features is different than another orientation feature.

6. The lead of claim 1 wherein the at least first orientation feature is deployable from a collapsed configuration to an expanded configuration.

7. The lead of claim 1 further comprising a dissolvable coating covering at least a portion of the at least first orientation feature.

8. The lead of claim 1 wherein the at least first orientation feature is elongated.

9. The lead of claim 1 wherein the at least first orientation feature is adjacent a flexibility transition region of the lead body.

10. The lead of claim 1 wherein the lead body is pre-shaped with a curved region, and the at least first orientation feature is adjacent the curved region.

11. The lead of claim 10 wherein the at least first orientation feature is adjacent an inflection region of the lead body.

12. The lead of claim 1 wherein the at least first orientation feature is integrally formed in the lead body.

13. A left ventricular lead for placement in a branch vessel of the coronary sinus, the vessel having a vessel wall and an adjacent myocardium, the lead comprising:
a lead body having a lumen extending therethrough;
at least a first electrode on the lead body; and
at least a first orientation feature protruding from the lead body for orienting a distal tip of the lead body into a selected branch vessel.

14. The lead of claim 13 wherein the at least first orientation feature is adjacent an inflection region of the lead body.

15. The lead of claim 13 wherein the at least first orientation feature is adjacent an outer tangent of the inflection region.

16. The lead of claim 13 wherein the at least first orientation feature is one of a tine, a leaf spring, a polymer protrusion, an expandable member, a balloon, a stent, a cage, or a shape memory alloy.

17. A method of implanting a lead in a selected branch vessel of the coronary sinus that is adjacent a myocardium, the method comprising:
providing a lead body having a lumen extending therethrough, at least a first electrode on the lead body; and at least a first orientation feature protruding from the lead body for orienting one or more of the electrodes into contact with the myocardium;
advancing the lead body through the coronary sinus and into the selected branch vessel; and
engaging the at least first orientation feature against a vessel wall of the branch vessel opposite the at least first electrode.

18. The method of claim 17 further comprising deploying the at least first orientation feature from a collapsed configuration to an expanded configuration.

19. The method of claim 17 further comprising engaging the at least first orientation feature against a surface of the heart to select a branch vessel.

20. The method of claim 17 further comprising engaging the at least first orientation feature against the vessel wall to fix the lead in a selected position.
